(19) United States
(12) Patent Application Publication
(10) Pub. No.: US 2016/0235667 A1
(43) Pub. Date: Aug. 18, 2016

(54) METHOD AND APPARATUS FOR MAKING COMPOSITIONS FOR PULMONARY ADMINISTRATION

(71) Applicant: VECTURA LIMITED, Chippenham, Wiltshire (GB)

(72) Inventor: MATTHEW GREEN, WILTSHIRE (GB)

(21) Appl. No.: 15/026,318

(22) PCT Filed: Oct. 1, 2014
(86) PCT No.: PCT/GB2014/052973

§ 371 Date: Mar. 31, 2016

(30) Foreign Application Priority Data
Oct. 2, 2013 (GB) 1317482.6
Oct. 2, 2013 (GB) 1317483.4

(51) Int. Cl. A61K 9/00; B01F 3/18; B01F 11/02; A61K 9/16; C07K 16/42

(52) U.S. Cl. CPC A61K 9/0075; A61K 9/1682; C07K 16/4291; A61K 9/1617; A61K 9/1623; B01F 11/02; B01F 3/18; C07K 2317/24; C07K 2317/21; C07K 2317/52; B01F 2215/0032; B01F 2215/0431; B01F 2215/044; B01F 2215/0454; B01F 2215/0477

(57) ABSTRACT

A method is disclosed for making a pharmaceutical composition for pulmonary administration comprising a pharmaceutically active protein or nucleic acid particle, the method comprising a step in which the inhalable pharmaceutically active protein or nucleic acid particle is acoustically blended in a resonant acoustic blender. The invention also relates to compositions for inhalation prepared by the method.

### BACKGROUND

The present invention relates generally to the field of mixing pharmaceutical powders. The apparatus is particularly suited for the field of inhalation.

Inhalation represents a very attractive, rapid and patient-friendly route for the delivery of systemically acting proteins or nucleic acids, as well as for proteins or nucleic acids that are designed to act locally on the lungs themselves. It is particularly desirable and advantageous to develop technologies for delivering drugs to the lungs in a predictable and reproducible manner.

The key features which make inhalation a useful protein or nucleic acid delivery route are: rapid speed of onset; improved patient acceptance and compliance for a non-invasive systemic route; reduction of side effects; product life cycle extension; improved consistency of delivery; access to new forms of therapy, including higher doses, greater efficiency and accuracy of targeting; and direct targeting of the site of action for locally administered drugs, such as those used to treat lung diseases.

However, the powder technology behind successful dry powders and dry powder inhaler (DPI) or pressured metered dose inhalers (pMDI) products remains a significant technical hurdle to those wishing to succeed with this route of administration and to exploit the significant product opportunities. Any suitable formulation must have properties that allow for the manufacture and metering of the powders, provide reliable and predictable resuspension and fluidisation, and avoid excessive retention of the powder within the dispensing device. One way of obtaining a resuspension and fluidisation involves mixing or blending of the formulations to be used in DPIs or pMDIs.

The mixing or blending of powders involves agitation resulting in the distribution of either heterogeneous or homogeneous particles to form the final formulation. Mixing processes are called upon in an attempt to effect a uniform distribution of particulates such as drug particles over a carrier particle.

Traditionally, mixing can be achieved in a variety of ways. Firstly, by a rotating shaft mounted impeller which is immersed in the fluid mixture or by tumbling the fluid mixture in a container vessel. Mixing may be continuous or intermittent.

Equipment, such as tumblers and tube blenders are well-used in the pharmaceutical industry often achieving blend uniformity after prolonged blending times. Unfortunately, segregation of these blends can readily occur especially during subsequent blend handling or transfer. The negative effects of segregation can include: uneven particle size or drug distribution, decreased flowability, reduced performance as well as changes in blend colour, taste, or appearance. Segregation is particularly prevalent when particles separate due to differences in their size, shape, or density. A further criticism levelled at traditional blending procedures, especially impeller blending processes, is that a substantial proportion of the blend is lost to the internal surfaces. This is a particular disadvantage for blends that contain expensive drugs. Traditional impeller processors are also known to generate heat within the blend, which may adversely affect the blend characteristics.

A major problem experienced by formulators is that uniform blends often take time to generate. This approach it is often associated with problems such as poor blend uniformity and undesirable heating of the constituent parts. Formulators face a delicate balance because over processing the formulation may change the blend dispersion characteristics thereby creating unwanted inter-batch variability. Conversely, under-processing may lead to the generation of API (Active Pharmaceutical Ingredient) “hotspots” which may not be detected by conventional blend uniformity tests. This is further complicated in the field of inhalation where not only is a uniform blend a prerequisite of a suitable formulation, but the dissociation of the active from the carrier must take place at a specific time in order to deliver a therapeutic dose to the patient. Uniform blends can be achieved using conventional machines but this often involves high energy blending and mixing procedures with rapid rotation speeds that impart undesirable effects to the powder such as, for example, heat, static or undesired milling of the particles.

Formulations that have heterogeneous particle size distributions are relatively easy to blend. Without wishing to be bound by theory, it is thought that the large particles create interparticulate spaces that permit the smaller particles to permeate into these spaces and thereby create a uniform blend. Homogeneous blends in the pharmaceutical sector are considered to be those with a coefficient of variation of less than 5%. Unlike heterogeneous particle blends, there is significant difficulty when attempting to blend two or more formulations that contain particles which are uniform in size and have a narrow particle size distribution. These homogenous formulations do not have the required interparticulate spaces that permit the smaller particles to permeate into these spaces and thereby create a uniform blend. These uniform and narrow particle size distributions are routinely obtained from apparatus such as spray driers.

Uniform blends with narrow particle size distributions are desirable in the field of inhalation because pharmaceutically active proteins or nucleic acids are highly potent molecules. A uniform blend with a narrow Particle Size Distribution (PSD) lends itself more easily to predictable pulmonary drug delivery.

Uniform particle blends of pharmaceutically active proteins or nucleic acids may be achieved by spray drying. A difficulty arises when attempting to blend two or more formulations each having similar particle sizes, for example attempting to blend two or more spray dried formulations. The constituent formulations, with similar particle sizes, do not have interparticulate spaces that lend themselves to easily obtaining blend uniformity. As a consequence, significant energy is required to achieve blend uniformity from two or more formulations each having similar particle sizes. This significant energy often involves long processing times accompanied by high shear and significant heat generation. These parameters, and in particular the heat generation, are to be avoided when blending protein or nucleic acid containing formulations.

In summary, the background art does not teach a system suitable for producing protein or nucleic acid formulations suitable for inhalation. Nor does the prior art teach a method for blending pharmaceutically active constituent parts of a formulation that are similar or uniform in size. What is needed is a rapid method for uniformly mixing protein or nucleic containing particulates in a manner that can be varied whilst still maintaining the physical structure of the fragile protein or nucleic acid and excipient materials within the pharmaceutical formulation.

### SUMMARY OF THE INVENTION

In view of the problems outlined above, the present application teaches the use of a resonant acoustic mixer for mixing pharmaceutically active protein or nucleic acid particles with advantageous blend homogeneities and aerosol performance.

The purpose of the invention is to provide a method of intimate processing of, for example, a plurality of fluids. These fluids may include liquid-liquid, solid-solid, liquid-solid or more than two fluid phases. One application is the mixing and dispersion of solids, in particular small homogenously sized particles. Other applications include preparing emulsions for pharmaceutical applications, accelerating physical and chemical reactions, for example biological reactions such as enzymatic processes, and suspending fine particles in fluids. The fluids referred to above may or may not include entrained solid particles. One application is the mixing and dispersion of fluids, for example solids, in particular small homogenously sized solid particles.

The present invention provides a method for mixing materials which afford minute control over mixing in a wide range of applications. The range of applications extends from bench scale formulations (up to 450 g) to large scale manufacture of pharmaceuticals (up to 420 kg). In one embodiment, the present invention provides a vibration mixer, driven by an electronically controllable motor or motors, adapted to allow control of the mixing process.

Yet another embodiment of the invention is a process to facilitate mixing by a selected frequency, amplitude or acceleration. Another embodiment of the invention is to disperse fine pharmaceutically active protein particles in a uniform manner throughout the formulation blend.

In one embodiment said pharmaceutically active protein or nucleic acid containing composition comprises a plurality of particles and said mixing step further comprises exposing said composition to a vibratory environment that is at a frequency between about 15 Hertz to about 1,000 Hertz and at an amplitude of between about 0.01 mm to about 50 mm thereby achieving micromixing of said composition.

A system and process for the application of acoustic energy to a reactor volume that can achieve a high level of uniformity of mixing is disclosed. The “micromixing” that is achieved and the effects in the combinations of frequency ranges, displacement ranges and acceleration ranges disclosed herein produce very high-quality blends, as defined by acceptable blend uniformity and constituent parts which exhibit improved physical character, for example aerosol performance and/or chemical stability and/or physical stability. This is especially noticeable when preparing delicate protein or nucleic acid systems.

The method disclosed herein can be practiced with the systems disclosed herein and with single mass vibrators, dual mass vibrators, and piezoelectric and magnetostrictive transducers.

Although some embodiments are shown to include certain features, the applicant(s) specifically contemplate that any feature disclosed herein may be used together or in combination with any other feature on any embodiment of the invention. It is also contemplated that any feature may be specifically excluded from any embodiment of an invention.

The invention relates, in one aspect, to a method for making a pharmaceutical composition, the method comprising a step in which pharmaceutically active protein or nucleic acid particles are acoustically blended in the presence of particles of an excipient material.

The invention relates, in one aspect, to a method for making a pharmaceutical composition, the method comprising a step in which a first formulation comprising pharmaceutically active protein particles is acoustically blended with a second formulation comprising pharmaceutically active protein particles.

The invention relates, in one aspect, to a method for making a pharmaceutical composition, the method comprising a step in which a first formulation comprising pharmaceutically nucleic acid particles is acoustically blended with a second formulation comprising pharmaceutically active nucleic acid particles.

A pharmaceutically active protein or nucleic acid is the substance in the pharmaceutical composition that is biologically active. The distinction between a pharmaceutically active protein or nucleic acid and excipient can be determined by referring to pharmaceutical reference literature.

Furthermore, an inhalable pharmaceutically active protein or nucleic acid must also have particle size distribution wherein D₁₀≦6 μm, D₅₀≦7 μm and D₉₀≦10 μm. Pharmaceutical formulations comprising particle size distribution wherein the D₉₀≧10 μm is not suitable for inhalation. This is because a substantial proportion of the protein or nucleic acid particles will impact higher up in the airways and in the oral-pharyngeal cavity. Considering the potency of these compounds, the delivery site must be precisely targeted to illicit the desired therapeutic effect. Pharmaceutically active protein or nucleic acid formulations with a non-inhalable drug component (D₉₀≧10 μm) should not be considered for safety reasons.

Without wishing to be bound by theory, the method of acoustically blending according to the present application provides for a homogenous mixing of material by an acoustic mixing method. The formulation is subjected to vibration at an amplitude and frequency that causes resonance of the particles within the formulation. When focused on a formulation, the acoustic energy converts into particle kinetic energy which, in isolation, is relatively insignificant. When the acoustic energy is focused on a population of particles the pockets of energised particles affected rapidly mix with surrounding particles due to the enlarged interparticulate spaces. This resonance causes macroscopic and microscopic turbulence within the blend enabling uniform mixing. Mixing using an acoustic blender is therefore quickly achieved without the use of impellors, blades, rotors, paddles or rotation of the containing vessel. Homogenous mixing of the pharmaceutical composition can be determined by a percentage coefficient of variation that is less than about 5%.

Similarly, when a suspension or bi-phasic liquid formulation is subjected to vibration at an amplitude and frequency this causes resonance of the liquid formulation. When focused on the liquid formulation, the acoustic energy affects the surfaces of the liquids causing them to ripple. As the energy is increased the ripple becomes greater until protrusions or invaginations occur at the liquid surface. Eventually these protrusions are so extensive that they break away from the liquid formulation entirely to join the other liquid phase and thereby create a new liquid-liquid surface and so the process is repeated until a distinct liquid-liquid surface no longer exists but at a macroscopic level the “bi-phasic” liquid formulation has now become uniform in appearance. Mixing is therefore quickly achieved without the use of impellors, blades, rotors or paddles.

In any and all embodiments disclosed herein, the term pharmaceutically active protein does not include any one of: palivizumab, interferon, adamalysin, serralysin, astacin, aerugen, Tumour Necrosis Factor Inhibitor (TNF inhibitors) and/or alpha 1-antitripsin.

In any and all embodiments disclosed herein, the term pharmaceutically active protein does not include any one of: dactinomycin, famiciclovir, palivizumab, interferon, caspofungin, capreomycin, vancomycin, ONO 6126, adamalysin, serralysin, astacin, Epithelial Sodium Channel Inhibitors P-680 and/or alpha 1-antitripsin.

Any acoustic apparatus suitable for the dissolution or destruction of biological cells is not suitable for use with any aspect of the invention, for example cell lysis sonication systems.

### DETAILED DESCRIPTION OF INVENTION

An embodiment of the invention is to facilitate acoustic mixing of two or more solids. Another embodiment of the invention is to facilitate acoustic mixing of one or more solids and one or more gases. Another embodiment of the invention is to facilitate acoustic mixing of one or more solids with one or more liquid particles. A further embodiment of the invention is to facilitate acoustic mixing of one or more solid with one or more liquid particles with one or more gases.

Mixing gram (g) to kilogram (kg) amounts of the entire pharmaceutical composition is contemplated according to all embodiments. Mixing milligram (mg), nanogram (ng) and smaller amounts of pharmaceutical composition are impractical and therefore not suitable due to loss of the constituents to the vessel wall. However milligram (mg), nanogram (ng) and smaller amounts of pharmaceutically active protein or nucleic acid can readily be mixed with excipient and/or additive resulting in a larger blend in the order of gram (g) to kilogram (kg) scale. Likewise milligram (mg), nanogram (ng) and smaller amounts of a first pharmaceutically active protein or nucleic acid can readily be mixed with a second pharmaceutically active protein or nucleic acid and optionally an excipient and/or additive resulting in a larger blend in the order of gram (g) to kilogram (kg) scale. Similarly mixing tonnes of pharmaceutical composition are also impractical because of the difficulties associated with routinely obtaining homogenous blends. Blend homogeneity is particularly important in the field of pulmonary drug delivery.

Solids are mixed by adding acoustic energy so that micromixing is achieved. A vibratory environment operating at a frequency between about 15 Hz to about 1,000 Hz with an amplitude between about 0.01 mm to about 50 mm provides the necessary acoustic energy required to mix solids. The size of the solids can be nano-sized to much larger particles, for example micrometers. The acoustic energy provided to the particles directly acts on the formulation to produce mixing. Other processes use components such as propellers to produce fluid motion through eddies which then mix the media. These eddies are dampened by the media and thus the mixing is localized near the component creating them, for example the blades, rotors or paddles. Acoustic energy supplied to the media is not subject to the localization of input mentioned above because the entire mixing vessel volume is subjected to the energy at the same time.

Specific frequency ranges for operating the acoustic blender include from about 5 Hz to about 1,000 Hz, preferably 15 Hz to about 1,000 Hz, more preferably 20 Hz to about 800 Hz, more preferably 30 Hz to 700 Hz, more preferably 40 Hz to 600 Hz, more preferably 50 Hz to 500 Hz, more preferably 55 Hz to 400 Hz, more preferably 60 Hz to 300 Hz, more preferably 60 Hz to 200 Hz, more preferably 60 Hz to 100 Hz, more preferably 60 Hz to 80 Hz, more preferably 60 Hz to 75 Hz, most preferably from about 60 to 61 Hz. The selection of the resonant frequency is the most important criterion because acceleration, amplitude and intensity can be modified accordingly. The selection of less energetic parameters as illustrated with Formulation 1A below will require either extended duration of acoustic blending or the selection of more energetic parameters as illustrated in example 1 below.

Specific time ranges for operating the acoustic blender include from at least 10 seconds, at least 30 seconds, at least 1 minute, for at least 2 minutes, for at least 3 minutes, for at least 4 minutes, for at least 5 minutes, for at least 6 minutes, for at least 7 minutes, for at least 8 minutes, for at least 9 minutes, for at least 10 minutes, for at least 11 minutes, for at least 12 minutes, for at least 13 minutes, for at least 14 minutes, for at least 15 minutes, for at least 16 minutes, for at least 17 minutes, for at least 18 minutes, for at least 19 minutes, for at least 20 minutes, for at least 21 minutes, for at least 22 minutes, for at least 23 minutes, for at least 24 minutes, for at least 25 minutes, for at least 26 minutes, for at least 27 minutes, for at least 28 minutes, for at least 29 minutes or for up to 60 minutes or for up to 30 minutes. For the avoidance of doubt, blending periods of less than 30 seconds are less preferred because whilst homogenous blends can be achieved, they are not routinely achievable as determined by a percentage coefficient of variation that is greater than about 5%. When selecting short intervals (for example, ≦1 minute), the selection of greater mixing intensities will be required (for example, ≧40% Intensity). The specific time periods disclosed herein refer to periods in which resonance is imparted to the pharmaceutical composition. It is possible for the resonance blending to be interrupted whilst, for example, content uniformity of the pharmaceutical composition is established. Upon completion of the content uniformity assessment, resonance blending may be resumed. The total duration of resonance blending of the pharmaceutical composition or of its constituent parts will be understood to be the specific time period disclosed herein. It is important to establish once a coefficient of variation of less than 5% has been achieved because acoustic blending should then be stopped, or closely monitored if a coefficient of variation of less than 4%, or less than 3%, or less than 2% or less than 1% is desired. It is possible to impart too much acoustic energy for too long and produce a formulation wherein the blend is not homogenous (due to re-segregation) as determined by a percentage coefficient of variation that is greater than about 5%. Diligent monitoring of the blend's content uniformity during acoustic blending will ensure this (i.e. CV>5%) does not happen.

In one embodiment, a method is disclosed for making a pharmaceutical composition, the method comprising a step in which an inhalable pharmaceutically active protein or nucleic acid is acoustically blended with excipient material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which a first inhalable pharmaceutically active protein is acoustically blended with a second inhalable pharmaceutically active protein and optionally an excipient material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which a first inhalable pharmaceutically active nucleic acid is acoustically blended with a second inhalable pharmaceutically active nucleic acid and optionally an excipient material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and additive material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and additive material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and additive material, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and additive material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and additive material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and additive material, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from 5 Hz to about 1,000 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from 5 Hz to about 1,000 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from about 30 Hz to 75 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from about 30 Hz to 75 Hz for a period of at least for at least 2 minutes. Preferably, wherein the excipient material comprises an inert carrier, preferably a non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from about 30 Hz to 75 Hz for a period of at least for at least 2 minutes. Preferably, wherein the excipient material comprises an inert carrier, preferably a non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from about 30 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from about 30 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from about 30 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from about 60 Hz to 75 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from about 60 Hz to 75 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from about 60 Hz to 75 Hz for a period of at least for at least 2 minutes. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with excipient material and magnesium stearate, wherein the acoustic frequency operating range is from about 60 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein is acoustically blended with a second inhalable pharmaceutically active protein and optionally with excipient material and magnesium stearate, wherein the acoustic frequency operating range is from about 60 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active nucleic acid is acoustically blended with a second inhalable pharmaceutically active nucleic acid and optionally with excipient material and magnesium stearate, wherein the acoustic frequency operating range is from about 60 Hz to 75 Hz for a period of at least for at least 2 minutes until a coefficient of variation of less than 5% is achieved. Preferably, wherein the excipient material comprises an inert carrier, preferably non-reducing disaccharide, preferably sucrose and/or trehalose.

In one embodiment, a method is disclosed for making a pharmaceutical composition, the method comprising a step in which an inhalable pharmaceutically active protein or nucleic acid is acoustically blended with polymersomes.

Polymersomes are manufactured from synthetic polymers are a class of artificial vesicles that may enclose a solid and/or solution.

In one embodiment, a method is disclosed for making a pharmaceutical composition, the method comprising a step in which an inhalable pharmaceutically active protein or nucleic acid is acoustically blended with liposomes.

Liposomes are manufactured from naturally or synthetic lipids, are a class of vesicles that may enclose a solid and/or solution.

In one embodiment incorporation of a protein or nucleic acid containing solid into a liquid is enhanced by exposing the solid and liquid to a vibratory environment that is operative to vibrate the combination at a frequency of between about 15 Hz to about 1,000 Hz with amplitude between 0.01 mm to about 50 mm. Incorporation can be so complete it is approaching the theoretical maximum. By placing the fluid and solids in a vibratory environment and, as a result, providing acoustic energy to the media, the effect is to fluidize the mixture. In the process, micromixing is accomplished throughout the vessel while macro-mixing the product. Complete and thorough mixing is accomplished by the use of acoustic energy at previously unachievable solids loadings. Similarly, in one embodiment incorporation of a solid into a protein or or nucleic acid containing liquid is enhanced by exposing the solid and liquid to a vibratory environment that is operative to vibrate the combination at a frequency of between about 15 Hz to about 1,000 Hz with amplitude between 0.01 mm to about 50 mm. Incorporation can be so complete it is approaching the theoretical maximum.

One embodiment of the invention is to facilitate acoustic mixing of two or more protein containing liquids, for example two or more miscible liquids (a linctus), or for example two or more non-miscible liquids (emulsions or creams). Another embodiment of the invention is to facilitate acoustic mixing of one or more liquids and one or more gases. Another embodiment of the invention is to facilitate acoustic mixing of one or more liquids with one or more solid particles. A further embodiment of the invention is to facilitate acoustic mixing of one or more liquids with one or more solid particles with one or more gases.

One embodiment of the invention is to facilitate acoustic mixing of two or more nucleic acid containing liquids, for example two or more miscible liquids (a linctus), or for example two or more non-miscible liquids (emulsions or creams). Another embodiment of the invention is to facilitate acoustic mixing of one or more liquids and one or more gases. Another embodiment of the invention is to facilitate acoustic mixing of one or more liquids with one or more solid particles. A further embodiment of the invention is to facilitate acoustic mixing of one or more liquids with one or more solid particles with one or more gases.

Liquid to liquid mixing is enhanced when a protein or a nucleic acid containing composition that comprises a plurality of liquids is exposed to a vibratory environment that vibrates the composition at a frequency between about 15 Hz to about 1,000 Hz with an amplitude between about 0.01 mm to about 50 mm. Liquids that are not miscible are readily mixed when subjected to this condition. Normal boundary layers which prevent mixing are broken and the liquids are freely and evenly distributed within each other. Micromixing with generation of micron to 100 micron droplets is achieved in this vibratory environment. The uniformity of droplet size and distribution is enhanced by this vibratory process thereby achieving greater mass transport, but the mixture is easily separated when the vibratory agitation is removed. Tuning the process between a frequency between about 15 Hz to about 1,000 Hz with an amplitude between about 0.01 mm to about 50 mm optimizes the transfer of acoustic energy into the fluid. This energy then generates an even distribution of droplets (larger than those generated with typical related processes) which collide with each other to affect mass transfer from one droplet to another. After the acoustic energy is removed, the liquids easily and quickly separate thus effecting high mass transfer without creating an emulsion.

One embodiment of the invention is to facilitate acoustic mixing of two or more protein containing pastes or protein containing suspensions. Another embodiment of the invention is to facilitate acoustic mixing of one or more pastes and one or more gases. Another embodiment of the invention is to facilitate acoustic mixing of one or more pastes with one or more solid particles. A further embodiment of the invention is to facilitate acoustic mixing of one or more pastes with one or more solid particles with one or more gases. Acoustic mixing of pastes comprising single or multiple inhalable pharmaceutically active protein/s may be to be dried before milling and then adding the micronized product into a final formulation. A distinct advantage of acoustic mixing is that viscosities from 1 cP to greater than 1,000,000 cP can be effectively mixed.

One embodiment of the invention is to facilitate acoustic mixing of two or more nucleic acid containing pastes or nucleic acid containing suspensions. Another embodiment of the invention is to facilitate acoustic mixing of one or more pastes and one or more gases. Another embodiment of the invention is to facilitate acoustic mixing of one or more pastes with one or more solid particles. A further embodiment of the invention is to facilitate acoustic mixing of one or more pastes with one or more solid particles with one or more gases. Acoustic mixing of pastes comprising single or multiple inhalable pharmaceutically active nucleic acid/s may be to be dried before milling and then adding the micronized product into a final formulation. A distinct advantage of acoustic mixing is that viscosities from 1 cP to greater than 1,000,000 cP can be effectively mixed.

The acoustic blender may be used to create protein or a nucleic acid containing emulsions such as those described above and this apparatus can readily be connected to spray drying systems or nebulisation systems to produce spray dried particles. In one embodiment a volatile material is acoustically blended with a second material containing active material, for example a pharmaceutically active material. During the course of spray drying the volatile material migrates to the surface of the droplet containing the active material. After volatilisation of the volatile material during the spray drying process, the protein or nucleic acid containing particle is left which has multiple dimples (resembling a golf ball) or connected holes (resembling a practice golf ball) on the surface or combinations thereof. The acoustic blender is highly efficient at minimizing the size of the volatile material, which in turn dictates the size of the holes or dimples in the final protein containing product. Volatile materials are those know to the person skilled in the art and importantly will be selected, used and treated with an abundance of caution when spray drying.

The acoustic blender may be used to create protein or nucleic acid containing suspensions such as those described above and this apparatus can readily be connected to spray drying systems or nebulisation systems to produce spray dried particles. A volatile material is acoustically blended with a second material containing active material, for example a pharmaceutically active material. During the course of spray drying the volatile material migrates to the surface of the droplet containing the active material. After volatilisation of the volatile material during the spray drying process, a particle is left which has multiple dimples (resembling a golf ball) or connected holes (resembling a practice golf ball) on the surface or combinations thereof. The acoustic blender is highly efficient at minimizing the size of the volatile material, which in turn dictates the size of the holes or dimples in the final product. Volatile materials are those know to the person skilled in the art and importantly will be selected, used and treated with an abundance of caution when spray drying.

In one embodiment the acoustic blender may be used to create protein containing suspensions such as those described above for use in a pMDI.

In one embodiment the acoustic blender may be used to create nucleic acid containing suspensions such as those described above for use in a pMDI.

In an alternate embodiment, the acoustic mixer contains a plurality of fixed deagglomerators for example a plurality of fixed sieves within the deagglomeration chamber. The sieves may have varying mesh sizes for example 63 μm, 90 μm, 125 μm, 150 μm, 212 μm etc. Most pharmaceutical powders can be sieved quickly with a standard sieve; however, some pharmaceutical powders have irregular-shaped particles or are cohesive, which can cause mesh-blinding due to problematic particles obstructing the aperture of the mesh. Screen blinding is a common problem when sieving difficult powders, typically those particles with a size of 175 μm and below. Screen blinding occurs when either one or a combination of problematic particles rest on or in an aperture of the mesh and stays there, or particles simply attach to the mesh wires occluding the aperture. When screen blinding occurs, the size of the particles falling to the next stack is then reduced. Alternatively, in the case of complete occlusion, it prevents particles from passing through these openings entirely. When screen blinding occurs, the useful screening area is reduced and, therefore, sieving capacity drops. When protein or nucleic acid containing powders are mixed according to this embodiment, the sieves screens act as either a barrier to preclude mixing of certain particles or the screen acts to facilitate the deagglomeration and blending process.

In another embodiment unsieved lactose may be added on top of a sieve screen within the acoustic mixer. Protein or nucleic acid containing particles and additive may reside below the sieve screen and the process results in a one-step sieving and blending process. The height of the screen can be manipulated to avoid any pharmaceutically active protein or nucleic acid entering the unscreened lactose held by the screen.

In an alternate embodiment, the acoustic mixer contains a plurality of compartments with shared walls along the length of the chamber of the acoustic mixer. Each compartment is designed to hold its own formulation constituent with associated sieve screen size. For example the first compartment may contain unsieved carrier particles with its dedicated screen size, the second compartments may contain unsieved excipient particles with its dedicated screen size and a third compartments may contain unsieved pharmaceutically active protein or nucleic acid particles with its own dedicated screen size. In an alternate embodiment, a compartment of the chamber of the acoustic mixer may contain a combination of these materials.

In an alternate embodiment, the acoustic mixer contains multiple containers with separate formulations to be mixed at the same time. This affords the convenience of avoiding cross contamination. Similarly in the event formulation components require separate conditioning, this can be achieved until the final protein or nucleic acid containing formulation needs to be assembled.

Traditional blending approaches require the presence of layers of material of one layer (n) placed upon the other (m) to form n/m/n/m/n etc. In one embodiment, the protein or nucleic acid containing blends produced do not require ordered layering (sandwiching) of the materials in order to achieve a homogenous blend as determined by the coefficient of variation and acceptable aerosol performance impaction analysis.

In one embodiment, method of the invention will, if the acoustic mixer is suitably arranged, produce composite active protein particles or nucleic acid. The inhalable composite active protein or nucleic acid particles are very fine particles of pharmaceutically active protein material which have, upon their surfaces, an amount of additive material. In one embodiment the additive material is in the form of a coating on the surfaces of the particles of pharmaceutically active material. The coating may be a discontinuous coating. The additive material may be in the form of particles adhering to the surfaces of the particles of active material.

During the acoustic mixing, particles of pharmaceutically active and additive material collide against each other with enough energy to locally heat and soften, break, distort, flatten and wrap the additive particles around the core active particle to form a particulate coating of additive on the active particle. The energy is generally sufficient to break up agglomerates but negligible size reduction of both components may occur. Unlike a blending or mixing process, in one embodiment, the method involves high energy parameters combined within a confined space which maximises the number high energy collisions between the particles resulting in a particulate coating of additive on the pharmaceutically active protein or nucleic acid containing particle.

Unlike the traditional blending or mixing process disclosed herein, in one embodiment, a method is disclosed for making composite protein particles for use in a pharmaceutical composition for pulmonary administration, the method comprising acoustically milling protein or nucleic acid particles in the presence of particles of an additive material. This process affords sufficient energy to the particles to sufficiently break-up any agglomerates of either protein or nucleic acid particles and additive material, and ensure an even distribution of the particulate additive material over the protein particles, and so that the particles of additive material become fused to the surface of the protein particles, wherein the additive material may be suitable for the promotion of the dispersal of the composite protein or nucleic acid particles upon actuation of an inhaler, wherein the acoustic milling step comprises adherent particles of additive material and blending these with protein or nucleic acid particles.

Alternatively, composite active particles may be made by acoustically blending protein or nucleic acid material with hollow microspheres. The hollow microspheres may be those referred to in Pharmaceutical Research, Vol. 25, No. 5, May 2008. The hollow microspheres are acoustically blended with pharmaceutically active protein or nucleic acid containing particles that are less than 2 μm, less than 1 μm, less than 0.5 μm and less than 0.25 μm. In one embodiment, a composite particle for use in a pharmaceutical composition for pulmonary administration, the composite particle comprising a hollow porous microsphere particle enveloping a pharmaceutically active protein or nucleic acid containing particle, the composite particles having a D₉₀≦10 μm. The advantage of acoustically blending hollow porous microsphere particles with a pharmaceutically active protein or nucleic acid containing particle is that the acoustic mixer is efficient at filling the microsphere with active but delicate enough not to destroy the structure of the hollow porous microsphere and thereby retain the benefits of these aerodynamically light particles. The vibration of the pharmaceutically active protein or nucleic acid containing particles with the hollow microsphere in close proximity enables the fine active to engage with the holes located on the surface of the microsphere and percolate into the hollow microsphere.

Alternatively, composite pharmaceutically active protein or nucleic acid containing particles may be created by acoustically blending a paste containing pharmaceutically active protein or nucleic acid material with hollow microspheres. The paste permeates the hollow microspheres assisted by the acoustic blending. In one embodiment, a composite pharmaceutically active protein or nucleic acid containing particle for use in a pharmaceutical composition for pulmonary administration, the composite particle comprising a hollow porous microsphere particle enveloping a paste or suspension, the composite particles having a D₉₀≦10 μm.

Alternatively, composite active particles may be made by acoustically blending pharmaceutically active protein or nucleic acid with multiple additives. In one embodiment, the composite pharmaceutically active protein or nucleic acid particles are created by sequentially adding an additive to the blend until a uniform coating of the active particles is achieved. In one embodiment, a composite particle for use in a pharmaceutical composition for pulmonary administration is disclosed, the composite particle comprising an pharmaceutically active protein or nucleic acid particle enveloped with layers of additive particle, the composite particles having a D₉₀≦10 μm and wherein the layers are 1 layer of additive, at least 1 layer of additive, 2 layers of additive, 3 layers of additive, or at least 3 layers of additive on the active particle.

This intensive process creates composite pharmaceutically active protein or nucleic acid particles for use in a pharmaceutical composition for pulmonary administration, each composite pharmaceutically active protein or nucleic acid particle comprising a particle of protein containing material and a particle of additive material on the surface of that particle of protein or nucleic acid containing material, wherein the composite protein containing particles have a D₉₀≦15 μm, ≦10 μm, ≦7 μm or ≦5 μm and wherein the additive material promotes the dispersion of the composite active particles upon actuation of a delivery device.

In one embodiment the additive particle is softer than the pharmaceutically active protein particle as measured by indentation hardness outlined in Alderborn and Nyström, Pharmaceutical Powder Compaction Technology, 1996. In this embodiment, the skilled person will understand that the absolute indentation hardnesses need not be determined precisely, merely that a qualitative assessment of additive hardness against the hardness of pharmaceutically active protein particle is required.

In one embodiment the additive particle is of equivalent particle size distribution to the pharmaceutically active protein or nucleic acid particle, as measured by D₅₀. Alternatively, the additive particle is of a smaller particle size distribution than the pharmaceutically active protein particle, in particular D₅₀ or alternatively, the additive particle is of a larger size than the active particle, in particular D₅₀. Alternatively, the sizes referred to above may be mass median aerodynamic diameters.

In one embodiment the first pharmaceutically active protein particle is of equivalent particle size distribution to the second pharmaceutically active protein particle, as measured by particular D₅₀. Alternatively, the first pharmaceutically active protein particle is of a smaller particle size distribution than the second pharmaceutically active protein particle, as measured by D₅₀ or alternatively, the first pharmaceutically active protein particle is of a larger size than the second pharmaceutically active protein particle, as measured by particular D₅₀. Alternatively, the sizes referred to above may be mass median aerodynamic diameters. Equivalent particle size distributions will be understood to vary by up to 100% based upon the particular D₅₀ values. For example the first pharmaceutically active protein particle formulation may have a D₅₀ of 5 μm and the second pharmaceutically active protein particle formulation may have a D₅₀ of 8 μm but neither formulations having a particle size distribution wherein the D₉₀≧10 μm.

In one embodiment the first pharmaceutically active nucleic acid particle is of equivalent particle size distribution to the second pharmaceutically active nucleic acid particle, as measured by particular D₅₀. Alternatively, the first pharmaceutically active nucleic acid particle is of a smaller particle size distribution than the second pharmaceutically active nucleic acid particle, as measured by D₅₀ or alternatively, the first pharmaceutically active nucleic acid particle is of a larger size than the second pharmaceutically active nucleic acid particle, as measured by particular D₅₀.

Alternatively, the sizes referred to above may be mass median aerodynamic diameters. Equivalent particle size distributions will be understood to vary by up to 100% based upon the particular D₅₀ values. For example the first pharmaceutically active nucleic acid particle formulation may have a D₅₀ of 5 μm and the second pharmaceutically active nucleic acid particle formulation may have a D₅₀ of 8 μm but neither formulations having a particle size distribution wherein the D₉₀≧10 μm. In one embodiment a first formulation comprising pharmaceutically active protein particles are of equivalent particle size distribution to a second formulation comprising pharmaceutically active protein particles, as measured and demonstrated by D₁₀, D₅₀ and D₉₀ values, especially when calculated using the span equation below. Equivalent particle size distribution is considered wherein the span number for each formulation is less than 150, more preferably less than 125, more preferably less than 100, or more preferably less than 50 prior to acoustically blending together.

In one embodiment a first spray dried formulation comprising pharmaceutically active protein particles are of equivalent particle size distribution to a second spray dried formulation comprising pharmaceutically active protein particles, as measured and demonstrated by D₁₀, D₅₀ and D₉₀ values, especially when calculated using the span equation below. Equivalent particle size distribution is considered wherein the span number for each formulation is from 1 to 30, more preferably from 1.1 to 20, more preferably from 1.2 to 10, or more preferably from 1.3 to 5 prior to acoustically blending together in a resonant acoustic blender.

In one embodiment a first formulation comprising pharmaceutically active nucleic acid particles are of equivalent particle size distribution to a second formulation comprising pharmaceutically active nucleic acid particles, as measured and demonstrated by D₁₀, D₅₀ and D₉₀ values, especially when calculated using the span equation below. Equivalent particle size distribution is considered wherein the span number for each formulation is less than 150, more preferably less than 125, more preferably less than 100, or more preferably less than 50 prior to acoustically blending together.

In one embodiment a first spray dried formulation comprising pharmaceutically active nucleic acid particles are of equivalent particle size distribution to a second spray dried formulation comprising pharmaceutically active nucleic acid particles, as measured and demonstrated by D₁₀, D₅₀ and D₉₀ values, especially when calculated using the span equation below. Equivalent particle size distribution is considered wherein the span number for each formulation is from 1 to 30, more preferably from 1.1 to 20, more preferably from 1.2 to 10, or more preferably from 1.3 to 5 prior to acoustically blending together in a resonant acoustic blender.

$$Span = \frac{{D}_{v 0.9} - {D}_{v 0.1}}{{D}_{v 0.5}}$$

Alternatively, composite pharmaceutically active protein or nucleic acid particles made using intensive milling techniques may be added to the acoustic mixer for assembly into a final blend. Suitable milling methods are those involving the Mechano-Fusion, TRV, Hybridiser and Cyclomix instruments. In one embodiment, the milling step involves the compression of the mixture of active and additive particles in a gap (or nip) of fixed, predetermined width (for example, as disclosed in WO 2002/43701). With all the intensive milling techniques disclosed above, the skilled person will appreciate the particular sensitivity of the pharmaceutically active proteins to both heat and mechanical sheer and modify their processes accordingly and also employ punctuated protein integrity assessments.

Low shear mixing applications are necessary to prevent or reduce damage to pharmaceutical formulations. This is achieved by placing the pharmaceutical formulations in a vibratory environment that is operated to vibrate the pharmaceutical formulations at a frequency of about 5 Hz to about 1,000 Hz with an amplitude between about 0.01 mm to about 50 mm. The pharmaceutical formulations are physically mixed with gases, solids and liquids in an environment of low shear and minimal particle to particle collisions. Particles are prevented from agglomerating into large agglomerates.

In one embodiment, the acoustic mixer contains dampeners within the formulation. These dampeners are designed to modify and absorb the energy entering the formulation thereby avoiding damaging delicate pharmaceutically active protein or nucleic acid particles within the formulation. These dampeners may be balloons, hollow balls, light polystyrene particles or any similar particle. These dampeners may be recovered from the formulation by sieving when required.

Intrusion or infusion of gases entrained into a solid media is enhanced by placing the solid media in an environment that is operative to vibrate the solid media at a frequency of about 5 Hz to about 1,000 Hz with an amplitude between 0.01 mm to about 50 mm. Boundary layers are broken and gases are forced into, out of and through the particulate structure.

Another embodiment of the invention is to cause vapour to permeate through the fluidised powder bed. In one embodiment, the acoustic mixer is connected to a conditioning apparatus, for blending and conditioning the formulation (or constituents thereof prior to assembling the formulation). In one aspect, the pharmaceutically active protein or nucleic acid may be conditioned under conditions of low relative humidity whilst the acoustic mixer is in operation. In one embodiment, the active is treated under conditions of less than 10% relative humidity whilst the acoustic mixer is in operation. In one embodiment, the pharmaceutically active protein or nucleic acid is treated under conditions of between 0.5% and 10% relative humidity, in one embodiment between 2% and 9%, in one embodiment between 3% and 8%, in one embodiment between 4% and 7%, in one embodiment between 4% and 6%, or in one embodiment less than 5%, whilst the acoustic mixer is in operation.

In one aspect, a method is disclosed, for making a pharmaceutical composition, the method comprising a step in which an inhalable pharmaceutically active protein or nucleic acid is acoustically blended by exposure to reduced level of relative humidity as compared to ambient conditions, wherein the acoustic frequency operating range is from 5 Hz to about 1,000 Hz for a period of at least 2 minutes. Acceptable conditioning may be determined by a sustained D₉₀≦20 μm for more than 1 week, preferably more than 1 month, preferably more than 3 months or more preferably more than 9 months.

In one aspect, the pharmaceutically active protein or nucleic acid may be conditioned under a humid atmosphere whilst the acoustic mixer is in operation. In one embodiment, the pharmaceutically active protein or nucleic acid is conditioned under a relative humidity ranging from 5 to 90%. When intending to process under conditions of higher humidity, relative humidity ranges from 50 to 90%, 55 to 87%, 60 to 84%, 60 to 80%, 65 to 80%, 70 to 75% or 70 to 80% are preferred. In one aspect, the pharmaceutically active protein or nucleic acid may be conditioned under conditions of higher humidity, relative humidity that ranges from 51 to 100%, 61 to 100%, 71 to 100%, 81 to 100% or 91 to 100% are suitable embodiments. When intending to process under conditions of reduced humidity, ranges are from 5 to 50%, 7.5 to 40%, 10 to 30%, 12.5 to 20% and in one embodiment less than 15% relative humidity are suitable. In the case of cryogenic preparation, for example with the use of liquid nitrogen, reduced humidity ranges will be less than 5%.

In one aspect, a method is disclosed, for making a pharmaceutical composition, the method comprising a step in which an inhalable pharmaceutically active protein or nucleic acid is acoustically blended by exposure to elevated level of relative humidity as compared to ambient conditions, wherein the acoustic frequency operating range is from 5 Hz to about 1,000 Hz for a period of at least 2 minutes. Acceptable conditioning may be determined by a sustained D₉₀≦10 μm for more than 1 week, preferably more than 1 month, preferably more than 3 months or more preferably more than 9 months.

In one aspect, the pharmaceutically active protein or nucleic acid may be conditioned under a solvent containing atmosphere, such as an organic solvent whilst the acoustic mixer is in operation. Solvents include alcohols and/or acetone. The skilled artisan would appreciate the nature of risk associated with processing under such environments. Suitable environments include ethanol/nitrogen in ratios of 5:95% (w/w), in one embodiment 2.5:97.5% (w/w) in one embodiment 1:99% (w/w). Alternatively methanol/nitrogen in ratios of 5:95% (w/w), in one embodiment 2.5:97.5% (w/w) in one embodiment 1:99% (w/w) may be used. Alternatively acetone/nitrogen in ratios of 5:95% (w/w), in one embodiment 2.5:97.5% (w/w) in one embodiment 1:99% (w/w) may be used. The solvent may be introduced as a vapour within the gas lines to the acoustic mixer. The solvent may be introduced as a vapour in increasing amounts, from ambient for a length of time, for example, then increasing or decreasing by not more than 5% (w/w), not more than 10% (w/w), not more than 15% (w/w), not more than 20% (w/w) or alternatively not more than 25% (w/w) from the initial baseline and then optionally returning the vapour amount to baseline whilst the acoustic mixer is in operation. Alternatively, the solvent may be introduced as a vapour in increasing amounts, from 0% for a length of time, for example, then increasing by 1% (w/w) increments whilst the acoustic mixer is in operation until a desired vapour concentration is achieved. Alternatively, once a steady vapour state is achieved the solvent vapour may be decreased within the vessel with processing time, either during operation of the acoustic mixer or afterwards. Humidity may also be varied over time during the treatment of the active ingredient. The length of time to which the particles are exposed to this humidity may also be varied.

When used herein, “water” is neither an excipient nor an additive material.

Conditioning of the formulation or its constituent parts may take place before, during and/or after operating the acoustic mixer.

In another aspect the acoustic mixing may take place in a vacuum. In another aspect the acoustic mixing may take place under a pressurised environment.

Another embodiment of the invention is to accelerate physical and chemical reactions. A further embodiment of the invention is to accelerate heat transfer away from a heat-sensitive pharmaceutically active protein or nucleic acid. Another embodiment of the invention is to accelerate mass transfer. Yet another embodiment of the invention is to suspend and distribute particles. A further embodiment of the invention is to distribute particles. Another embodiment of the invention is to cause micromixing.

In another aspect the pharmaceutically active protein or nucleic acid is conditioned at a maximum temperature whilst the acoustic mixer is in operation. In one embodiment, the temperature is at not more than 30° C., in one aspect not more than 35° C., in one aspect not more than 40° C., in one aspect not more than 50° C., or not more than 60° C. Processing temperatures may be controlled for example via an external or integrated cooling jacket. Alternatively, the processing temperature may also be controlled via a suitably heated or cooled atmosphere introduced into the mixing chamber. Alternatively, temperature may also be varied over time during the treatment of the active ingredient. For example the heated atmosphere may be introduced by increasing temperature with processing time until the desired temperature is achieved. Alternatively, once a steady heated state is achieved the temperature may be decreased within the vessel with processing time.

A particular advantage of blending with an acoustic mixer is that a minimal rise in temperature following formulation processing is obtained, even after extended processing periods. In one embodiment, the temperature rise following blending is no more than 5° C., in one aspect no more than 10° C., in one aspect no more than 15° C., in one aspect no more than 20° C., or in one aspect no more than 30° C. In each of these aspects blend completion is determined by a CV of less than 5%. In one embodiment, use of an additive material in a pharmaceutical composition for pulmonary administration, wherein the additive material is suitable for minimising an increase in blend temperature during blending as compared with the same blend and process in the absence of the additive material. Suitable additive materials for this purpose include magnesium stearate.

In order to determine the initial homogeneity, the quantity of pharmaceutically active protein or nucleic acid (as determined by, for example, HPLC) in each sample is expressed as a percentage of the original recorded weight of the powder sample. The values for all the samples are then averaged to produce a mean value, and the coefficient of variation (CV) around this mean is calculated. The coefficient of variation is a direct measure of the homogeneity of the mix. A powder, whose homogeneity measured as a percentage coefficient of variation, is less than about 5% can be regarded as acceptable and a coefficient of variation of 2% is excellent.

In one aspect the additive material is an anti-adherent material that will tend to decrease the cohesion between the pharmaceutically active protein containing particles, and between the pharmaceutically active protein containing particles and other particles present in the pharmaceutical composition, for example carrier particles or a second pharmaceutically active particle.

In one aspect the additive material is an anti-adherent material that will tend to decrease the cohesion between the pharmaceutically active nucleic acid containing particles, and between the pharmaceutically active nucleic acid containing particles and other particles present in the pharmaceutical composition, for example carrier particles or a second pharmaceutically active particle.

The additive material may be an anti-friction agent (glidant), suitably to give better flow of the pharmaceutical composition in, for example, a dry powder inhaler which will lead to a better dose reproducibility.

Where reference is made to an anti-adherent material, or to an anti-friction agent, the reference is to include those materials which are able to decrease the cohesion between the particles, or which will tend to improve the flow of powder in an inhaler, even though they may not usually be referred to as anti-adherent material or an anti-friction agent. For example, leucine is an anti-adherent material as herein defined and is generally thought of as an anti-adherent material but lecithin is also an anti-adherent material as herein defined, even though it is not generally thought of as being anti-adherent, because it will tend to decrease the cohesion between the active ingredients and between the active ingredient and other particles present in the pharmaceutical composition.

The additive material may be in the form of particles which tend to adhere to the surfaces of active ingredient, as disclosed in WO1997/03649. Alternatively, the additive material may be coated on the surface of the active ingredient by a co-milling method, as disclosed in WO2002/43701. Therefore, in one aspect of the invention, the method may further comprise and additional step of coating the surface of the active ingredient with an additive material (e.g. by a co-milling method).

The additive material may include one or more compounds selected from amino acids and derivatives thereof, and peptides and derivatives thereof. Amino acids, peptides and derivatives of peptides are suitably physiologically acceptable and give acceptable release of the active ingredient on inhalation.

The additive may comprise one or more of any of the following amino acids: leucine, isoleucine, lysine, valine, methionine, and phenylalanine. The additive may be a salt or a derivative of an amino acid, for example aspartame or acesulfame K. Alternatively, the additive consists substantially of an amino acid, or of leucine, advantageously L-leucine. The L-, D and DL-forms of an amino acid may also be used. As indicated above, leucine has been found to give particularly efficient dispersal of the active ingredient on inhalation.

The additive may include one or more water soluble substances. A water soluble substance may be a substance that may be capable of dissolving wholly or partly in water and which is not entirely insoluble in water. This may help absorption of the additive by the body if it reaches the lower lung. The additive may include dipolar ions, which may be zwitterions. It is also advantageous to include a spreading agent as an additive, to assist with the dispersal of the composition in the lungs. Suitable spreading agents include surfactants such as known lung surfactants (e.g. ALEC™) which comprise phospholipids, for example, mixtures of DPPC (dipalmitoyl phosphatidylcholine) and PG (phosphatidylglycerol). Other suitable surfactants include, for example, dipalmitoyl phosphatidyl than olamine (DPPE), dipalmitoyl phosphatidylinositol (DPPI).

The additive may comprise a metal stearate, or a derivative thereof, for example, sodium stearyl fumarate or sodium stearyl lactylate. Advantageously, it comprises a metal stearate, for example, zinc stearate, magnesium stearate, calcium stearate, sodium stearate or lithium stearate. In one embodiment, the additive material comprises magnesium stearate, for example vegetable magnesium stearate, or any form of commercially available metal stearate, which may be of vegetable or animal origin and may also contain other fatty acid components such as palmitates or oleates.

The additive may include or consist of one or more surface active materials. A surface active material may be a substance capable reducing the surface tension of a liquid in which it is dissolved. Surface active materials may in particular be materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof such as glyceryl behenate. Specific examples of such materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the additive may be cholesterol.

Other possible additive materials include sodium benzoate, hydrogenated oils which are solid at room temperature, talc, titanium dioxide, aluminium dioxide, silicon dioxide and starch. Also useful as additives are film-forming agents, fatty acids and their derivatives, as well as lipids and lipid-like materials.

In one aspect, additive particles are composed of lactose. The additive particles may be lactose fines. The additive lactose may be added a various stages of the formulation assembly or the additive lactose may be formed as a result of processing of a larger lactose carrier particle. Said processing cleaves off the protruding asperities and produces smaller lactose particles that may re-adhere to the larger carrier particles or combine with different components of the composition. When used as an additive, the lactose fines have a D₉₀≦20 μm, preferably ≦15 μm.

A particular advantage of magnesium stearate in acoustic powder blending is it minimises a rise in formulation temperature during processing with an acoustic mixer. The presence of magnesium stearate in the blend also maintains acceptable blend homogeneity as determined by the coefficient of variation and acceptable aerosol performance as determined by aerosol impaction analysis. In one aspect, additive particles comprise magnesium stearate.

In one aspect a plurality of different additive materials can be used. In one embodiment combinations of additive materials include lactose fines and magnesium stearate. In one embodiment the lactose fines and magnesium stearate are in loose association. Alternatively, the magnesium stearate is smeared or fused over the particles of fine lactose as a composite excipient particle.

Carrier particles may be of any acceptable inert excipient material or combination of materials. For example, carrier particles frequently used in the prior art may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, dextrose or lactose. In one embodiment, the carrier particles are composed of lactose. Suitable examples of such excipient include LactoHale 300 (Friesland Foods Domo), LactoHale 200 (Friesland Foods Domo), LactoHale 100 (Friesland Foods Domo), PrismaLac 40 (Meggle), InhaLac 70 (Meggle).

Alternatively, composite carrier particles may be made by acoustically blending carrier material with additive and optionally pharmaceutically active protein particles. In one embodiment, the composite carrier particles are created by sequentially adding an additive to the blend until a coating of the carrier particles is achieved. In one embodiment, a composite carrier particle for use in a pharmaceutical composition for pulmonary administration, the composite particle comprising a carrier particle enveloped with a layer of additive particle, the composite particles having a diameter of greater than 63 μm and wherein the layers are 1 layer of additive, at least 1 layer of additive, 2 layers of additive, 3 layers of additive, or at least 3 layers of additive on the carrier particle. A composition comprising particles falling within the scope of this embodiment will easily recover these particles via a 63 μm sieve screen.

Alternatively, composite carrier particles may be made by acoustically blending carrier material with pharmaceutically active protein particles. In one embodiment, the composite carrier particles are created by sequentially adding an active to the blend until a coating of the carrier particles is achieved. In one embodiment, a composite carrier particle for use in a pharmaceutical composition for pulmonary administration, the composite particle comprising a carrier particle enveloped with a layer of additive particle, the composite particles having a diameter of greater than 63 μm and wherein the layers are 1 layer of active, at least 1 layer of active, 2 layers of active, 3 layers of active, or at least 3 layers of active on the carrier particle. A composition comprising particles falling within the scope of this embodiment will easily recover these particles via a 63 μm sieve screen. In one embodiment, the layers may comprise alternate layers of active. For example, additive 1 coated by additive 2 which is in turn coated by additive 1.

The ratio in which the carrier particles (if present) and pharmaceutically active protein or nucleic acid particles are mixed will depend on the type of inhaler device used, the type of pharmaceutically active protein or nucleic acid particles used and the required dose. The carrier particles may be present in an amount of at least 50%, at least 70%, at least 90% and at least 95% based on the combined weight of the active ingredient and the carrier particles and additives, if additive is present.

Wet granulation is a process in which a mix of powders is agglomerated with a liquid binder forming larger particles or granules. These granules normally have a size distribution in the range of 100 μm to 2000 μm, and are mainly used for tablet compaction and capsule filling. Wet granulation is typically used to improve the flow, compressibility and homogeneity of the mixture used to produce solid dosage forms. The most widely used excipients for granulation are microcrystalline cellulose, lactose and dibasic calcium phosphate. The three main types of wet granulation process are (i) low shear granulation using a planetary mixer, (ii) high shear granulation using a high speed mixer with an impeller and chopper and (iii) fluid-bed granulation using fluid-bed drier.

These granulated lactose particles are particularly useful in inhalable formulations because they have a multitude of clefts and crevices in which the drug particles may reside. However, they require delicate blending approaches to avoid damaging their fragile structures. This has meant that until now, these shear sensitive granulated lactose particles required prolonged blending times at lower energy levels to maintain their physical structure. Surprisingly we have found that blends comprising granulated lactose particles can be achieved in much shorter periods of times whilst still possessing acceptable blend homogeneity as determined by the coefficient of variation, acceptable aerosol performance as determined by aerosol impaction analysis and still maintain their physical size as determined by microscopy and particle size analysis.

In one embodiment the use of an acoustic blender for the preparation of a pharmaceutical composition wherein the pharmaceutical composition possesses at least equivalent or better blend homogeneity, at least equivalent or better aerosol performance as compared with the same starting formulation processed by a TRV blender (GEA Pharma Systems) but wherein the blend homogeneity is obtained in less than 90%, less than 80%, less than 70%, less than 60%, less than 50%, less than 40%, less than 30%, less than 20% or less than 10% of the blend time taken by the TRV blender, wherein the composition is an inhalable composition for treatment of respiratory diseases.

In one embodiment the use of an acoustic blender for the preparation of a composition comprising pharmaceutically active protein or nucleic acid particles is disclosed wherein the pharmaceutical composition possesses at least equivalent or better blend homogeneity, at least equivalent or better aerosol performance as compared with the same starting formulation processed by a Diosna but wherein the blend homogeneity is obtained in less than 90%, less than 80%, less than 70%, less than 60%, less than 50%, less than 40%, less than 30%, less than 20% or less than 10% of the blend time taken by the Diosna, wherein the composition is an inhalable composition for treatment of respiratory diseases.

Alternatively, composite carrier particles may be made by acoustically blending pharmaceutically active protein particles onto the carrier particles. In one embodiment, alternate layers of a first pharmaceutically active protein particle followed by a second pharmaceutically active protein material followed by the first pharmaceutically active protein may be used to coat the carrier particles.

Alternatively, composite carrier particles may be made by acoustically blending pharmaceutically active nucleic acid particles onto the carrier particles. In one embodiment, alternate layers of a first pharmaceutically active nucleic acid particle followed by a second pharmaceutically active nucleic acid material followed by the first pharmaceutically active nucleic acid may be used to coat the carrier particles.

In one embodiment, composite carrier particles are created by sequentially adding an additive to the blend until a uniform coating of the carrier particles is achieved. In one embodiment, a composite particle for use in a pharmaceutical composition for pulmonary administration, the composite particle comprising an carrier particle enveloped with layers of additive particle, the composite carrier particles having a diameter of more than 50 μm and wherein the layers are 1 layer of additive, in one embodiment at least 1 layer of additive, in one embodiment 2 layers of additive, in one embodiment 3 layers of additive, or in one embodiment at least 3 layers of additive on an active particle and then optionally adding pharmaceutically active protein or nucleic acid particles.

An alternative embodiment provides a pharmaceutically active protein or nucleic acid particle for use in a pharmaceutical composition, a pharmaceutical composition for inhalation, in one embodiment a powder for a dry powder inhaler. In one embodiment, the active ingredient may be for use in a pharmaceutical composition for a pressurized metered dose inhaler (pMDI).

In another embodiment of the present invention, powders in accordance with the present invention may be administered using active or passive devices. In one embodiment of the invention, the inhaler device is an active device, in which a source of compressed gas or alternative energy source is used. Examples of suitable active devices include Aspirair™ (Vectura), Microdose™ and the active inhaler device produced by Nektar Therapeutics (as covered by U.S. Pat. No. 6,257,233).

In an alternative embodiment, the inhaler device is a passive device, in which the patient's breath is the only source of gas which provides a motive force in the device. Examples of “passive” dry powder inhaler devices include the Rotahaler™ and Diskhaler™ (GlaxoSmithKline) and the Turbohaler™ (AstraZeneca), Monohaler™ (Miat), GyroHaler™ (Vectura) and Novolizer™ (Viatris GmbH).

The size of the doses can vary from nanograms to micrograms to milligrams, depending upon the pharmaceutically active protein or nucleic acid, the delivery device and disease to be treated. Suitably the dose will range from 1 ng to 50 mg of active ingredient, in one embodiment 10 mg to 20 mg and in one embodiment 100 μg to 10 mg. The skilled artisan will appreciate that dose of the active will depend on the nature of the active pharmaceutical ingredient, therefore a dose of 1 mg to 10 mg, in one embodiment 2 mg to 8 mg, in one embodiment 3 mg to 7 mg and in one embodiment 4 mg to 5 mg is required. Alternatively a dose of 5 mg to 15 mg, a dose of 6 mg to 14 mg, in one embodiment 7 mg to 13 mg and in one embodiment 8 mg to 12 mg is required. Alternatively a dose of 10 mg to 20 mg, in one embodiment 12 mg to 18 mg, in one embodiment 14 mg to 16 mg and in one embodiment 14.5 mg to 15.5 mg is required. Alternatively a dose of 20 mg to 25 mg, more preferably in one embodiment 21 mg to 24 mg, in one embodiment 22 mg to 23 mg and in one embodiment 22.5 mg is required. Doses referred to above are nominal doses. These amounts should not be confused with the total amount of the pharmaceutical composition that is prepared.

Reference to doses herein is generally a reference to metered doses (MD) (or nominal doses (ND), the two terms may be used interchangeably). The MD is the dose of active pharmaceutical ingredient in the blister or capsule or formulation holding receptacle prior to delivery to the patient.

The emitted dose (ED) or delivered dose (DD) (the two terms may be used interchangeably) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise but still acceptable).

The fine particle dose (FPD) is the total mass of active agent which is emitted from the device following actuation which is present in an aerodynamic particle size smaller than a defined limit. This limit is generally taken to be 5 μm MMAD if not expressly stated to be an alternative limit, such as 3 μm, 2 μm or 1 μm, etc.

The fine particle fraction (FPF) is normally defined as the FPD (the dose that is <5 μm MMAD) divided by the delivered Dose (DD) which is the dose that leaves the device. The FPF is expressed as a percentage. Herein, the FPF of DD is referred to as FPF (DD) and is calculated as FPF (DD)=(FPD/DD)×100%.

The fine particle fraction (FPF) may also be defined as the FPD divided by the Metered Dose (MD) which is the dose in the blister or capsule, and expressed as a percentage. Herein, the FPF of MD is referred to as FPF (MD), and may be calculated as FPF (MD)=(FPD/MD)×100%.

According to an embodiment of the present invention, a receptacle is provided, holding a dose of the pharmaceutically active protein or nucleic acid prepared according to the present invention. The receptacle may be a capsule or blister, or a foil blister.

Pharmaceutically active protein or nucleic acid, suitably in the form of a powder, in accordance with the present invention may be pre-metered. The powders may be kept in foil blisters which offer chemical and physical protection whilst not being detrimental to the overall performance. Indeed, the formulations thus packaged tend to be stable over long periods of time, which is very beneficial, especially from a commercial and economic point of view.

In one embodiment, the composition according to the present invention is held in a receptacle containing a single dose of the powder, the contents of which may be dispensed using one of the aforementioned devices. Reservoir devices may also be used.

The invention also relates to a method of acoustically processing an pharmaceutically active protein, the method comprising submitting a pharmaceutically active protein to vibrational processing in the absence of another powder material, optionally then combining the active ingredient with another agent, such as another active ingredient, optionally a second pharmaceutically active protein, an excipient and/or additive, and then packaging the active ingredient into a receptacle or drug delivery device.

The invention also relates to a method of acoustically processing an pharmaceutically active nucleic acid, the method comprising submitting a pharmaceutically active nucleic acid to vibrational processing in the absence of another powder material, optionally then combining the active ingredient with another agent, such as another active ingredient, optionally a second pharmaceutically active nucleic acid, an excipient and/or additive, and then packaging the active ingredient into a receptacle or drug delivery device.

In one aspect the pharmaceutically active protein or nucleic acid may also have been subjected to compression and shearing forces in the absence of another powder material, taking care not to totally destroy the biological activity. When employing this approach, some reduction in biological activity is acceptable.

In one embodiment of the present invention there is provided a composition, in one embodiment a pharmaceutical composition, comprising a pharmaceutically active protein or nucleic acid made by a method according to the present invention in combination with an additional ingredient such as an additive, carrier and/or flavouring agent or other excipient.

The use of an acoustic mixer in the context of a formulation blend confers a number of distinct advantages. Firstly, the absence of agitators blades or impellers in the mixing chamber minimizes and destruction of delicate structures within the blend. Unlike the localised mixing produced by blades and impellors an acoustic mixer provides a uniform shear field throughout the mixing chamber. The use of an acoustic mixer avoids “dead zones” in the mixing chamber where efficient mixing does not take place. This is particularly useful when attempting to obtain uniform blends. The acoustic mixing chamber can be used as the shipping container. This affords the benefit of conducting the mixing process in one location and shipping the entire blend to a completely new location, where, for example, a powder filling line may be located in a different country. This benefit is particularly useful when a blend must be filled into capsules or blisters because intermittent agitation may be employed during the blister/capsule filling process. This intermittent agitation avoids problems such as blocking of the hopper caused by “rat holes” within the powder blend. The term “rat hole” describes the phenomenon wherein powder particles form temporary bridges thereby holding the formulation above the bridge in place whilst the formulation below the bridge collapses creating a formulation cavity. One of the key technical challenges in manufacturing a powder blend is the inability to migrate from laboratory bench scale through to commercial scale. This obstacle is encountered because the particle physics relevant to laboratory bench scale do not translate to a commercial scale arrangement. Due to the advantage of a uniform shear field throughout the mixing chamber irrespective of the scale use, the scale up procedures are more straightforward. Finally, the most distinct benefit of the acoustic mixer is the benefit of shorter blending times compared with traditional Turbula or Tumble mixers.

In one embodiment, a method is disclosed for making a pharmaceutical composition, the method comprising a step in which an inhalable pharmaceutically active protein or nucleic acid is acoustically blended with excipient material, wherein the acoustic frequency operating range is from 5 Hz to about 1,000 Hz for a period of at least 2 minutes until a coefficient of variation of less than 5% is achieved and wherein the acoustically blending vessel containing the blended pharmaceutical composition may then attach to an automated filling apparatus. Preferably, wherein the excipient material comprises lactose.

Any acoustic apparatus suitable for the dissolution or destruction of biological cells is not suitable for use with any aspect of the invention, for example cell lysis sonication systems.

Proteins are complex organic macromolecules that contain carbon, hydrogen, oxygen, nitrogen, and usually sulphur. Proteins may also contain metal ions, such as iron. Examples of metal containing proteins include myoglobin and zinc hexameric insulin. Proteins are composed of one or more chains of amino acids. Proteins are fundamental components of all living cells and include many substances, such as enzymes, hormones, and antibodies.

Proteins are high molecular weight compounds. They consist of at least one multiple chain of amino acid residues linked by peptide bonds and are folded into a specific three-dimensional shape usually containing alpha helices and beta sheets as well as looping and folded chains maintained by further chemical bonding. The presence or peptide (amide) bond within a molecule does not make the molecule a protein.

A protein is a molecule comprising polypeptides, has a non-homogeneous charge distribution across the molecule and has three-dimensional domain structure as a consequence of non-covalent bonds.

As a subset of pharmaceutically active proteins, antibodies (also known as immunoglobulins) may be used in formulations to be processed by acoustic mixing according to the invention. Antibodies present as five isotypes namely IgA, IgD, IgE, IgG and IgM in placental mammals. In addition, the following molecules will also be considered as antibodies capable for use with the invention, namely: Domain antibody (dAb), fragment crystallizable region (Fc region), single-chain variable fragment (scFv), bispecific monoclonal antibody (BsMAb or BsAb), Recombinant Chimeric Antibody (hCAb), single-domain antibody (sdAb), bispecific antibody fragments (bsFab), bispecific antibody fragments (bsFab′₂), Single-chain variable fragment (scFv), tandem Single-chain variable fragment (scFv), Diabody, Single-chain Diabody or Minibody as discussed in Carter, Experimental Cell Research, 1261-1269 (2011) and Chames, British Journal of Pharmacology, 220-233 (2009).

The following antibodies illustrate the invention:

Omalizumab (IgG1) for asthma, ALX-0171 (Trimeric Nanobody) for Respiratory tract disease, Reslizumab (IgG4) for asthma, Mepolizumab (IgG1) for asthma and or COPD, Benralizumab (IgG1) for asthma and or COPD, Brodalumab (IgG2) for asthma, Secukinumb (IgG1) for asthma, Lebrikizumab (IgG4) for asthma, Tralokinumab (IgG4) for asthma, Dupilumab (IgG4) for asthma, FG3019 (IgG1) for Idiopathic Pulmonary Fibrosis, STX-100 for Idiopathic Pulmonary Fibrosis, SAR156597 (tetravalent bispecific tandem immunoglobulin) for Idiopathic Pulmonary Fibrosis, Canakinumab (IgG1) for COPD, MEDI-557 (IgG1) for Respiratory tract disease, Freolimumab (IgG4) for Idiopathic Pulmonary Fibrosis and/or Cetuximab (IgG1) for lung cancer;

The antibody or antibody fragment comprising at least one Fab molecule, wherein the light chain variable region, V_{L} and the heavy chain region, V_{H} of the Fab molecule are linked by one or more disulfide bonds, and use of the same in treatment or prophylaxis as disclosed in WO2011117648, the text of which is hereby incorporated by reference;

The antibody Fab fragments in which the heavy chain constant region terminates at the interchain cysteine of C_{H}1. Also provided are antibody Fab fragments in which the heavy chain constant region terminates at the interchain cysteine of C_{H}1 to which one or more effector molecules are attached as disclosed in WO2005003169, the text of which is hereby incorporated by reference;

Antibody molecules having specificity for antigenic determinants of human IL-13, therapeutic uses of the antibody molecules and methods for producing said antibody molecules as disclosed in WO2010103274, the text of which is hereby incorporated by reference. In a specific embodiment, an antagonistic antibody which binds human IL-13 comprising a heavy chain, wherein the variable domain of the heavy chain comprises the sequence given in Sequence Identity Number 1 for CDR-H1, the sequence given in Sequence Identity Number 2 for CDR-H2 and the sequence given in Sequence Identity Number 3 for CDR-H3 and additionally comprising a light chain, wherein the variable domain of the light chain comprises the sequence given in Sequence Identity Number 4 for CDR-L1, the sequence given in Sequence Identity Number 5 for CDR-L2 and the sequence given in Sequence Identity Number 6 for CDR-L3 is disclosed in WO2010103274, the text of which is hereby incorporated by reference;

An antagonistic anti-human IL-13 antibody or antigen-binding fragment thereof that binds specifically to human IL-13, wherein said antibody competitively inhibits binding of an antibody produced by hybridoma 228B/C-1 which is designated with the ATCC deposit number PTA-5657 to IL-13 as disclosed by WO2005062967, the text of which is hereby incorporated by reference;

An isolated neutralising human, humanised or chimeric antibody that binds to IL-13, wherein the isolated human antibody binds to human IL-13 with a KD of less than 55 pM, wherein said KD is determined via a solution-based Biacore or KinExA assay and wherein (i) the antibody specifically binds to a polypeptide consisting of amino acids 20-29 of SEQ ID NO:96; or (ii) the antibody binds to residues 21-33 or 70-80 of SEQ ID NO:72; or (iii) the antibody comprises the amino acids of SEQ ID NO:50 in the heavy chain and comprises the amino acids of SEQ ID NO:52 in the light chain; or (iv) the antibody comprises the amino acids of SEQ ID NO:38 in the heavy chain and comprises the amino acids of SEQ ID NO:40 in the light chain; or v. the antibody comprises the amino acids of CDR1, CDR2 and CDR3 of SEQ ID NO:50 in the heavy chain, as shown in Table 18 and comprises the amino acids of CDR1, CDR2 and CDR3 of SEQ ID NO:52 in the light chain, as shown in Table 20; or (vi) the antibody comprises the amino acids of CDR1, CDR2 and CDR3 of SEQ ID NO:38 in the heavy chain, as shown in Table 18; and comprises the amino acids of CDR1, CDR2 and CDR3 of SEQ ID NO:40 in the light chain, as shown in Table 20 of WO2006055638, the text of which is hereby incorporated by reference;

An antibody to IL-13 or an IL-13 binding fragment thereof which is present in an amount sufficient to decrease the lung inflammation and/or tissue fibrosis in a subject, thereby treating a lung inflammation and/or tissue fibrosis in said subject.

As a building block of pharmaceutically active proteins, pharmaceutically active peptides may be used in formulations to be processed by acoustic mixing.

A compound may be classified as a peptide if it is composed of up to 10 the same or different amino acids. A molecule comprising 10 or more amino acids in the backbone is considered as a “polypeptide”. A polypeptide can present as a secondary structure (alpha helix, beta-sheet) without further folding into domains.

Nucleic acids are complex organic macromolecules. Nucleic acids, which include DNA (deoxyribonucleic acid) and RNA (ribonucleic acid), are made from nucleotide monomers. Nucleic acid molecules range in size from several nucleotides such as small interfering RNA (siRNA) to large chromosomes. Chromosomes are not considered as suitable for inhalation and consequently should not be considered as pharmaceutically active nucleic acids

The invention further relates to an inhalable pharmaceutical composition comprising a plurality of pharmaceutically active peptide particles wherein in the blend homogeneity (% RSD) is less than 5.0, less than 4.0, or preferably less than 3.0, or preferably less than 2.0 or preferably less than 1.0. Optionally wherein the pharmaceutically active peptide particles comprise a first pharmaceutically active peptide and at least a second pharmaceutically active peptide. Optionally wherein the pharmaceutically active peptides reside in different particles and are acoustically blended in a resonant acoustic blender. Optionally wherein the pharmaceutically active peptides reside in the same particle and are acoustically blended in a resonant acoustic blender. Optionally wherein the pharmaceutically active peptide particles are obtained by spray drying.

The invention further relates to an active ingredient obtainable or obtained using the above method.

The invention further relates to an inhaler device comprising a pharmaceutically active protein obtainable or obtained by the method of the invention, or an active ingredient which has been further processed where necessary into a pharmaceutically acceptable form.

The invention further relates to a receptacle, such as a blister or capsule, comprising a dose of an active ingredient, obtainable or obtained by the method of the invention, or an active ingredient which has been further processed where necessary into a pharmaceutically acceptable form.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or an when used in conjunction with the term “comprising” in the claims and/or the specification may mean “one,” but it is also consistent with the meaning of “one or more,” “at least one,” and “one or more than one.” The use of the term or in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

While certain embodiments of the present invention are described in detail above, the scope of the invention is not to be considered limited by such disclosure, and modifications are possible without departing from the spirit of the invention as evidenced by the examples and claims.

The present invention is illustrated by the by the experimental data set out below, which is not limiting upon the invention.

In one embodiment a method is disclosed wherein the acoustic blending step is carried out in the presence of a liquid, preferably wherein the liquid comprises a propellant suitable for use in a pressurised metered dose inhaler device.

In one embodiment a method is disclosed wherein the pharmaceutically active protein is conditioned during the acoustic blending, preferably wherein the pharmaceutically active protein is conditioned by an elevated level of relative humidity as compared to ambient conditions. wherein the pharmaceutically active protein is conditioned by increasing the relative humidity over time to about 60-80% RH, preferably to about 75%. Optionally, wherein the minimum temperature is at least 10° C., at least 20° C., at least 30° C., at least at least 40° C., at least 50° C., preferably at least 60° C. and the pharmaceutically active protein still retains at least 50% of the original biological activity.

In one embodiment a method is disclosed wherein the pharmaceutically active protein is conditioned during the acoustic blending,

In one embodiment a method is disclosed for making a pharmaceutical composition for pulmonary administration comprising an inhalable pharmaceutically active nucleic acid, the method comprising a step in which the inhalable pharmaceutically active nucleic acid is acoustically blended in a resonant acoustic blender. In one embodiment the nucleic acid is DNA. In one embodiment the nucleic acid is RNA. In one embodiment the nucleic acid is formulated as a dry powder, preferably wherein the nucleic acid is formulated as a particle. In one embodiment the nucleic acid is formulated as a dry powder.

In one embodiment for making a pharmaceutical composition for pulmonary administration comprising an inhalable pharmaceutically active nucleic acid, the nucleic acid is spray dried.

In one embodiment for making a pharmaceutical composition for pulmonary administration comprising an inhalable pharmaceutically active nucleic acid, the acoustic blending is conducted at from 5 Hz to about 1,000 Hz, preferably 15 Hz to about 1,000 Hz, more preferably 20 Hz to about 800 Hz, more preferably 30 Hz to 700 Hz, more preferably 40 Hz to 600 Hz, more preferably 50 Hz to 500 Hz, more preferably 55 Hz to 400 Hz, more preferably 60 Hz to 300 Hz, more preferably 60 Hz to 200 Hz, more preferably 60 Hz to 100 Hz, more preferably 60 Hz to 80 Hz, more preferably 60 Hz to 75 Hz, most preferably from about 60 to 61 Hz.

In one embodiment for making a pharmaceutical composition for pulmonary administration comprising an inhalable pharmaceutically active nucleic acid, the acoustic blending is conducted for at least 1 minute, for at least 2 minutes, for at least 3 minutes, for at least 4 minutes, for at least 5 minutes, for at least 6 minutes, for at least 7 minutes, for at least 8 minutes, for at least 9 minutes, for at least 10 minutes, for at least 11 minutes, for at least 12 minutes, for at least 13 minutes, for at least 14 minutes, for at least 15 minutes, for at least 16 minutes, for at least 17 minutes, for at least 18 minutes, for at least 19 minutes, for at least 20 minutes, for at least 21 minutes, for at least 22 minutes, for at least 23 minutes, for at least 24 minutes, for at least 25 minutes, for at least 26 minutes, for at least 27 minutes, for at least 28 minutes, for at least 29 minutes or for up to 30 minutes, or for up to 60 minutes.

In one embodiment for making a pharmaceutical composition for pulmonary administration comprising an inhalable pharmaceutically active nucleic acid, the pharmaceutical composition further comprises an excipient material, preferably wherein the excipient material is particulate, preferably the excipient is a non-reducing disaccharide, preferably sucrose and/or trehalose, preferably wherein the non-reducing disaccharide has a D₁₀≦250 μm, D₅₀≦500 μm and D₉₀≦800 μm, more preferably wherein in the D₁₀≦5-15 μm, D₅₀≦60-80 μm and D₉₀≦120-160 μm, most preferably D₅₀≦15 μm, D₅₀≦80 μm and D₉₀≦160 μm.

In one embodiment for making a pharmaceutical composition for pulmonary administration comprising an inhalable pharmaceutically active nucleic acid, the pharmaceutical composition further comprises additive material, preferably wherein the additive material is particulate, preferably wherein the additive material comprises either an amino acid, a phospholipid, a polymersome or a liposome. The additive material is present in an amount of about 0.1 to about 5% (w/w), preferably from about 1 to about 5% (w/w), preferably about 0.1 to 4% (w/w), preferably about 0.1 to 3% (w/w), preferably about 0.1 to 2% (w/w), preferably about 0.1 to 1% (w/w), more preferably about 0.1 to 0.5% (w/w) of the pharmaceutical composition.

In one preferred embodiment for making a pharmaceutical composition for pulmonary administration comprising an inhalable pharmaceutically active nucleic acid, the pharmaceutical composition the additive material comprises a metal stearate, preferably wherein the metal stearate is either magnesium stearate or calcium stearate, preferably wherein the additive is magnesium stearate.

In one embodiment for making a pharmaceutical composition for pulmonary administration comprising an inhalable pharmaceutically active nucleic acid, the acoustic blending step is carried out in the presence of a liquid, preferably wherein the liquid comprises a propellant suitable for use in a pressurised metered dose inhaler device.

In one embodiment for making a pharmaceutical composition for pulmonary administration comprising an inhalable pharmaceutically active nucleic acid, the composition is conditioned during the acoustic blending, wherein the pharmaceutically active nucleic acid is conditioned by an elevated level of relative humidity as compared to ambient conditions, wherein the pharmaceutically active nucleic acid is conditioned by increasing the relative humidity over time to about 60-80% RH, preferably to about 75%.

In one embodiment for making a pharmaceutical composition for pulmonary administration comprising an inhalable pharmaceutically active nucleic acid, the pharmaceutically active nucleic acid is conditioned at a minimum temperature, wherein the minimum temperature is at least 10° C., at least 20° C., at least 30° C., at least at least 40° C., at least 50° C., preferably at least 60° C. and the pharmaceutically active nucleic acid still retains at least 50% of the original biological activity, wherein the conditioned pharmaceutically active nucleic acid has a reduced amorphous content as compared with the starting material.

In one embodiment for making a pharmaceutical composition for pulmonary administration comprising an inhalable pharmaceutically active nucleic acid, the pharmaceutically active nucleic acid, is micronised prior to acoustic blending, preferably cryogenic micronisation. Alternatively the micronisation is by impact milling or jet milling, preferably air-jet milling, more preferably cryogenic jet milling.

In one embodiment for making a pharmaceutical composition for pulmonary administration comprising an inhalable pharmaceutically active nucleic acid, wherein after acoustic blending the pharmaceutical composition is packaged into a receptacle or delivery device.

In one embodiment for making a pharmaceutical composition for pulmonary administration comprising an inhalable pharmaceutically active nucleic acid, wherein the pharmaceutical composition is for localised pulmonary administration, preferably wherein the active is for localised effect, alternatively wherein the active is for systemic effect.

In one embodiment a pharmaceutical composition is disclosed for pulmonary administration comprising an inhalable pharmaceutically active nucleic acid, the pharmaceutical composition comprises a plurality of pharmaceutically active nucleic acid particles wherein in the blend homogeneity (% RSD) is less than 5.0, less than 4.0, or preferably less than 3.0, or preferably less than 2.0 or preferably less than 1.0, optionally wherein the pharmaceutically active nucleic acid particles comprise a first pharmaceutically active nucleic acid and at least a second pharmaceutically active nucleic acid, optionally wherein the pharmaceutically active nucleic acids reside in different particles and are acoustically blended in a resonant acoustic blender.

In one embodiment a pharmaceutical composition is disclosed for pulmonary administration comprising an inhalable pharmaceutically active nucleic acid, the pharmaceutical composition comprises a plurality of pharmaceutically active nucleic acid particles wherein the pharmaceutically active nucleic acids reside in the same particle and are acoustically blended in a resonant acoustic blender.

[symbol]

### EXAMPLES

Selected embodiments of the present invention will now be explained with reference to the examples. It will be apparent to those skilled in the art from this disclosure that the following descriptions of the embodiments are for illustration only and not for the purpose of limiting the invention as defined by the appended claims and their equivalents.

### Example 1

A batch of spray dried formulation (salbutamol sulphate 1.0% w/w, leucine 10.0% w/w and trehalose 89.0% w/w) was mixed with a spray dried carrier excipient formulation (amino sulfonic acid 99.5% w/w and leucine 0.5% w/w) using a resonant acoustic blender (LabRAM, Resodyn Corporation).

This was repeated using a range of intensities and mixing times (Table 1).

TABLE 1
Resonant acoustic mixer parameters for formulations A to F

| | | Time (minute) | | |
|---|---|---|---|---|
| | | 1 | 3 | 5 |
| Intensity | 30 | 1A | — | 1B |
| (%) | 40 | 1C | 1D | — |
| | 50 | 1E | — | 1F |

The formulations were manufactured to achieve a target API concentration in the final product of 0.01% w/w. This concentration was selected as it is a very dilute system which would best exemplify the homogenisation ability of the resonant acoustic blender (more dilute systems are much harder to homogenise).

The particle size of both of these components were assessed prior to mixing using the sympatec laser diffractor, RODOS, R4 lens, 1 bar dispersion pressure via ASPIROS, results for both formulations are shown in Table 2, both are within the inhalation range and are equivalent size distributions.

TABLE 2
Particle size analysis by Sympatec laser diffraction

| Formulation | D₁₀ (μm) | D₅₀ (μm) | D₉₀ (μm) |
|---|---|---|---|
| Spray dried Salbutamol | 0.9 | 2.7 | 6.1 |
| sulphate formulation | | | |
| Spray dried carrier | 0.9 | 2.5 | 5.7 |
| excipient formulation | | | |

The salbutamol sulphate formulation (0.2 g) was added to the carrier excipient (19.8 g) and mixed for 1 minute at 30% intensity. The formulation mixed well as determined by visual inspection (Formulation 1A).

The salbutamol sulphate formulation (0.2 g) was added to the carrier excipient (19.8 g) and mixed for 5 minutes at 30% intensity. The formulation mixed well as determined by visual inspection (Formulation 1B).

The salbutamol sulphate formulation (0.2 g) was added to the carrier excipient (19.8 g) and mixed for 1 minute at 40% intensity. The formulation mixed well as determined by visual inspection (Formulation 1C).

The salbutamol sulphate formulation (0.2 g) was added to the carrier excipient (19.8 g) and mixed for 3 minutes at 40% intensity. The formulation mixed well as determined by visual inspection (Formulation 1D).

The salbutamol sulphate formulation (0.2 g) was added to the carrier excipient (19.8 g) and mixed for 1 minute at 50% intensity. The formulation mixed well as determined by visual inspection (Formulation 1E).

The salbutamol sulphate formulation (0.2 g) was added to the carrier excipient (19.8 g) and mixed for 5 minutes at 50% intensity. The formulation mixed well as determined by visual inspection (Formulation 1F).

All formulations were subjected to content uniformity (CU) analysis (n=6 per formulation) (Table 3).

TABLE 3
Content uniformity results for salbutamol sulphate
resonant acoustic blender formulations

| | Mean % of Theoretical | |
|---|---|---|
| Formulation | drug content (% w/w) | % RSD |
| 1A | 108.81 | 10.9 |
| 1B | 99.29 | 3.1 |
| 1C | 102.48 | 3.9 |
| 1D | 96.11 | 2.6 |
| 1E | 99.93 | 1.0 |
| 1F | 103.94 | 0.7 |

Formulation 1A demonstrates that 30% mixing intensity for 1 minute provided insufficient energy to produce a formulation with acceptable content uniformity (CU) on this occasion. The CU data provides evidence that the resonant acoustic blender can successfully blend two powders of small particle size and achieve homogeneity of less than 1.0% RSD. To achieve a successful blend, close attention must be paid to the content uniformity (CU) of the manufacturing process.

### Example 2

A batch of spray dried Omalizumab formulation (trade name Xolair, Roche/Genentech and Novartis) was manufactured from 150 mg pre-filled syringe comprising approximately histidine (25 mM) and arginine (90 mM). The Omalizumab spray dried formulation consisted of Omalizumab 32.0% w/w, associated buffers 4.6% w/w, leucine 10.0% w/w and trehalose 53.4% w/w (Formulation 2A). was mixed with a portion of a carrier excipient (composed of spray dried amino sulfonic acid 99.5% w/w and leucine 0.5% w/w) (Formulation 2B) using the LabRAM resonant acoustic blender (Resodyn) at a range of intensities and mixing times outlined in Table 4.

TABLE 4
Omalizumab RAM formulation process parameters

| | | Time (minute) | | |
|---|---|---|---|---|
| | | 1 | 3 | 5 |
| Intensity | 30 | — | — | — |
| (%) | 40 | — | 2C | — |
| | 50 | 2D | — | 2E |

The formulations were manufactured to achieve a target API concentration in the final product of 1.2% w/w.

This concentration was selected as it is a very dilute system which would best exemplify the homogenisation ability of the resonant acoustic blender (more dilute systems are much harder to homogenise), it is higher than the salbutamol sulphate formulations due to the Omalizumab being more difficult to detect analytically.

The Omalizumab spray dried formulation (150 mg) was added to the carrier excipient (3850 mg) and mixed for 3 minutes at 40% intensity. The formulation mixed well as determined by visual inspection (Formulation 2C).

The Omalizumab spray dried formulation (150 mg) was added to the carrier excipient (3850 mg) and mixed for 1 minute at 50% intensity. The formulation mixed well as determined by visual inspection (Formulation 2D).

The Omalizumab spray dried formulation (150 mg) was added to the carrier excipient (3850 mg) and mixed for 5 minutes at 50% intensity. The formulation mixed well as determined by visual inspection (Formulation 2E).

The particle size of the input components and RAM formulations were assessed using the sympatec laser diffractor, RODOS, R4 lens, 1 bar dispersion pressure via ASPIROS, results are shown in Table 5. The input components are both are within the inhalation range and are equivalent size distributions.

TABLE 5
Particle Size Distribution for formulations 2A-2E

| Formulation | D₁₀ (μm) | D₅₀ (μm) | D₉₀ (μm) |
|---|---|---|---|
| 2A | 0.8 | 1.9 | 4.3 |
| 2B | 0.9 | 2.5 | 5.7 |
| 2C | 0.8 | 2.2 | 5.1 |
| 2D | 0.8 | 2.2 | 5.2 |
| 2E | 0.8 | 2.3 | 5.2 |

There are no significant changes in the PSD of the resonant acoustic blender processed formulations and they remain dispersible and within the inhalation range at a low dispersion pressure (1 bar).

### Content Uniformity

All resonant acoustic blender processed formulations were subjected to content uniformity (CU) analysis (n=10 per sample). Results are shown in Table 6.

TABLE 6
Omalizumab for formulation CU results

| Formulation | Mean % of Theoretical drug | |
|---|---|---|
| (batch number) | content (% w/w) | % RSD |
| 2C | 90.51 | 1.6 |
| 2D | 91.10 | 1.4 |
| 2E | 90.51 | 0.6 |

All formulations are homogenous and pass the acceptance criteria, the mean drug content is between 90.0%-110.0% of the target drug content and % RSD <5.0%.

The CU data provides evidence that the resonant acoustic blender can successfully process two powders of small particle size and achieve homogeneity of the protein.

TABLE 7
Omalizumab formulation GPC results

| | Monomer | | Other | | Other | | Other | |
|---|---|---|---|---|---|---|---|---|
| | Molecular | | Molecular | | Molecular | | Molecular | |
| Sample | Weight | Monomer | Weight | Other | Weight | Other | Weight | Other |
| Description | (kDa) | (%) | (kDa) | (%) | (kDa) | (%) | (kDa) | (%) |
| 2C | 142 | 95 | 713* | 3 | 48 | 1 | 14 | 1 |
| 2D | 144 | 98 | 454 | 1 | 42 | 1 | 16 | 1 |
| 2E | 143 | 96 | 475 | 4 | — | — | 17 | 0 |
| 2A | 143 | 99 | 1326* | 1 | 34 | 0 | 18 | 0 |

*A calculated approximation based on the molecular weights of known protein molecules used within the standard.

All formulations appear to have a similar profile by Gel Permeation Chromatography (GPC). There does not appear to be a significant difference between the formulations processed in the resonant acoustic blender and the spray dried pre-blend thereby demonstrating that processing by the resonant acoustic blender is not deleterious to the Omalizumab.

### ELISA

The activity of each formulation was assessed, alongside the Omalizumab spray dried formulation used in the resonant acoustic blender, results are shown in Table 8.

TABLE 8
ELISA results for Omalizumab formulations

| Formulation | Average activity (%) | | % RSD |
|---|---|---|---|
| 2A | High dilution | 88 | 2.36 |
| | Low dilution | 92 | 2.61 |
| 2C | High dilution | 107 | 10.97 |
| | Low dilution | 181* | 7.73 |
| 2D | High dilution | 103 | 14.66 |
| | Low dilution | 82 | 2.56 |
| 2E | High dilution | 122 | 18.97 |
| | Low dilution | 88 | 4.62 |

*Suspected dilution error.

TABLE 9
Non-Reducing SDS-PAGE

| | Monomer | | Other | | Other | |
|---|---|---|---|---|---|---|
| | Molecular | | Molecular | | Molecular | |
| | Weight | Monomer | Weight | Other | Weight | Other |
| Sample | (kDa) | (%) | (kDa) | (%) | (kDa) | (%) |
| Standard | 147 | 99 | 119 | 1 | ND | ND |
| 2A | 148 | 99 | 123 | 1 | ND | ND |
| 2C | 144 | 97 | 116 | 2 | 200 | 1 |
| 2D | 143 | 98 | 115 | 2 | ND | ND |
| 2E | 143 | 98 | 115 | 2 | ND | ND |

ND = Nothing detected

All formulations appear to have a similar profile by non-Reducing SDS-PAGE. There does not appear to be a significant difference between the formulations processed in the resonant acoustic blender and the spray dried pre-blend or the standard thereby demonstrating that processing by the resonant acoustic blender is not deleterious to the Omalizumab

TABLE 10
Reducing SDS-PAGE

| | Light | Heavy | | | | | | |
|---|---|---|---|---|---|---|---|---|
| | Chain | Chain | | | Other | | Other | |
| | Molecular | Molecular | Light | Heavy | Molecular | | Molecular | |
| | Weight | Weight | Chain | Chain | Weight | Other | Weight | Other |
| Sample | (kDa) | (kDa) | (%) | (%) | (kDa) | (%) | (kDa) | (%) |
| Standard | 29 | 48 | 27 | 69 | 236 | 3 | 78 | 1 |
| 2A | 28 | 47 | 28 | 68 | 228 | 3 | 78 | 1 |
| 2C | 29 | 48 | 28 | 69 | 227 | 3 | ND | ND |
| 2D | 28 | 47 | 28 | 69 | 222 | 3 | ND | ND |
| 2E | 28 | 47 | 28 | 68 | 222 | 3 | ND | ND |

ND = Nothing detected

All formulations appear to have a similar profile by reducing SDS-PAGE. There does not appear to be a significant difference between the formulations processed in the resonant acoustic blender, the spray dried pre-blend or the standard

### DSC

All spray dried and resonant acoustic blender processed formulations were assessed for their thermal properties using differential scanning calorimetry (DSC). Scan range 25-160° C. using a scan rate of 25° C.min⁻¹ in hermetically sealed pans.

All formulations have a glass transition temperature (Tg) ˜71° C. The heat capacity for this transition for the resonant acoustic blender formulations, compared to the Omalizumab spray dried formulation, is lower (0.4 J/(g.° C.) compared to ˜0.65 J/(g.° C.)) due to the presence of the crystalline carrier particles.

### Thermogravimetric Analysis

All spray dried and resonant acoustic blender processed formulations were assessed for their moisture content using thermogravimetric analysis (TGA). Scan range 25-180° C. scan rate of 25° C.min⁻¹. The Omalizumab formulation (2A) exhibits a moisture content of 2.8% LOD; this has been greatly reduced in the resonant acoustic blender formulations to 0.3% LOD.

### Aerosol Performance

The aerosol performance of each formulation was assessed using gravimetric Fast Screen Impactor (FSI), using the inhaler disclosed in international patent publication number WO 2010 086285 with a 12.5 mg fill weight. The resultant % FPF (fine particle fraction <5.0 μm) was calculated.

TABLE 11
Aerosol performance of formulations

| | Omalizumab | | | FPD | FPF as |
|---|---|---|---|---|---|
| | concen- | Omalizumab | Evacu- | (mass on | of fill |
| | tration | target dose | ation | filter) | mass |
| Formulation | (% w/w) | (mg) | % | (mg) | (%) |
| A | 1.2 | 0.15 | 84 | 3.50 | 30 |
| B | 1.2 | 0.15 | 87 | 3.54 | 28 |
| C | 1.2 | 0.15 | 88 | 3.24 | 26 |
| Omalizumab | 32.0 | 4.0 | 67 | 6.48 | 52 |
| spray dried | | | | | |
| formulation | | | | | |

The % FPF of all formulations manufactured using the resonant acoustic blender demonstrate the powder remains acceptably dispersible and aerosolisable.

1. A method for making a pharmaceutical composition for pulmonary administration comprising acoustically blending inhalable particles comprising a pharmaceutically active protein in a resonant acoustic blender, wherein the pharmaceutically active protein is not any one of palivizumab, interferon, Tumour Necrosis Factor Inhibitor, adamalysin, serralysin, astacin, aerugen and alpha 1-antitrypsin.

2. The method of claim 1, wherein the pharmaceutically active protein is not any one of dactinomycin, famiciclovir, caspofungin, capreomycin, vancomycin, ONO 6126, Epithelial Sodium Channel Inhibitors and P-680.

3. The method of claim 1, wherein the pharmaceutically active protein is an antibody selected from the group consisting of a chimeric antibody, a humanised antibody, and a human antibody.

4. The method of claim 1, wherein the pharmaceutically active protein is formulated as a dry powder.

5. The method of claim 1, wherein the pharmaceutically active protein is spray dried.

6. The method of claim 1, wherein the acoustic blending is conducted at from 5 Hz to about 1,000 Hz.

7. The method of claim 1, wherein the acoustic blending is conducted for at least 1 minute.

8. The method of claim 1, wherein the pharmaceutical composition further comprises an excipient material, wherein the excipient material is a non-reducing disaccharide.

9. The method of claim 1, wherein the pharmaceutical composition further comprises an additive material.

10. The method of claim wherein the non-reducing disaccharide has a D₁₀≦250 μm, D₅₀≦500 μm and D₉₀≦800 μm.

11. The method of claim 9 wherein the additive material is particulate, and wherein the additive material comprises at least one selected from the group consisting of an amino acid, a phospholipid, polymersome, a liposome and a metal stearate.

12. The method of claim 11, wherein the metal stearate is either magnesium stearate or calcium stearate.

13. The method according to claim 9, wherein the additive material is present in an amount of about 0.1 to about 5% (w/w) of the pharmaceutical composition.

14. The method of claim 1, further comprising mechanofusing the pharmaceutically active protein prior to acoustic blending.

15. The method of claim 1, further comprising micronising the pharmaceutically active protein prior to acoustic blending.

16. The method of claim 15, wherein the micronising is one selected from the group consisting of impact milling, jet milling, air-jet milling, and cryogenic jet milling.

17. The method of claim 1, further comprising packaging the pharmaceutical composition is packaged into a receptacle or delivery device after acoustic blending.

18. (canceled)

19. A pharmaceutical composition comprising a pharmaceutically active protein, obtained by the method of claim 1.

20. An inhaler device comprising the pharmaceutical composition obtained by the method of claim 1.

21-32. (canceled)
